(19) United States
(12) Patent Application Publication
(10) Pub. No.: US 2015/0045680 A1
(43) Pub. Date: Feb. 12, 2015

(54) BLOOD-PRESSURE RELATED INFORMATION DISPLAY DEVICE

(71) Applicants: OMRON Corporation, Kyoto (JP); OMRON HEAL THCARE Co., Ltd., Kyoto (JP)

(72) Inventors: Keisuke Yamada, Kyoto (JP); Chie Yamamoto, Kyoto (JP); Naoki Tsuchiya, Kyoto (JP); Mitsuo Kuwabara, Kyoto (JP)

(73) Assignees: OMRON HEALTHCARE CO., LTD., Kyoto (JP); OMRON CORPORATION, Kyoto (JP)

(21) Appl. No.: 14/385,467

(22) PCT Filed: Dec. 25, 2012
(86) PCT No.: PCT/JP2012/083511

§ 371 Date: Sep. 15, 2014

(30) Foreign Application Priority Data
Mar. 16, 2012 (JP) 2012-060280

(51) Int. Cl. A61B 5/021; A61B 5/00

(52) U.S. Cl. CPC A61B 5/021; A61B 5/742
USPC 600/485

(57) ABSTRACT

A blood pressure-related information display device displays a representative value setting field for setting a representative value obtainment mode as an image in a part of a display screen. The representative value setting field includes a setting area and a list area, the latter of which is displayed when, for example, a doctor clicks an inverted triangular mark in the setting area with a mouse. Representative value obtainment modes are displayed in the list area in list format so that all the modes can be viewed. The list area is configured so that any one of a plurality of types of representative value obtainment modes can be selected.

### TECHNICAL FIELD

This invention relates to blood pressure-related information display devices that display information related to a measurement subject's blood pressure in a display screen.

This invention also relates to programs for causing a computer to execute a method for displaying information related to a measurement subject's blood pressure in a display screen.

### BACKGROUND ART

At present, a blood pressure meter that stores a measured blood pressure value in a memory in association with measurement time information, measurement condition information, and so on, calculates average values for specific time periods such as a morning time period and an evening time period by averaging pluralities of measured blood pressure values, calculates a risk value based on results of calculating the average values, and displays the risk values is known as a device that displays information related to a measurement subject's blood pressure, as disclosed in Patent Literature 1 (JP 2004-261452A).

A data recording program that imports biological information into a personal computer through communication or manual input in order to support a measurement subject's health management is also known.

These systems (the blood pressure meter, the program, and so on) support health-related guidance for the measurement subject based on biological information such as a blood pressure value.

### CITATION LIST

### Patent Literature

Patent Literature 1: JP 2004-261452A

### SUMMARY OF INVENTION

However, with respect to the aforementioned conventional techniques, a blood pressure value used as the basis of the calculations cannot be selected according to a doctor's preferences, such as, for example, selecting only the second measured blood pressure value, selecting only the minimum value, or the like from the plurality of blood pressure values measured during the specific time period.

Accordingly, one or more embodiments of the claimed invention provide a blood pressure-related information display device that displays information related to a measurement subject's blood pressure in a display screen, and that enables blood pressure data used as the basis of a calculation to be easily selected according to a doctor's preferences from a plurality of pieces of measured blood pressure data.

One or more embodiments of the claimed invention also provide a non-transitory computer readable medium having a program stored thereon that causes a computer to execute a method for displaying information related to a measurement subject's blood pressure in a display screen, and that enables blood pressure data used as the basis of a calculation to be easily selected according to a doctor's preferences from a plurality of pieces of measured blood pressure data.

A blood pressure-related information display device according to one or more embodiments of the claimed invention is a blood pressure-related information display device that displays information related to a measurement subject's blood pressure in a display screen, and includes: a blood pressure data obtainment unit that obtains blood pressure data of the measurement subject for each day in a given period; a representative value obtainment mode setting unit for identifying a first instance from when the blood pressure data was measured first in a given day to after a predetermined amount of time has passed or an instance from when the blood pressure data was measured last in a given day to a predetermined amount of time before that last measurement, presenting, as a list in the display screen, a plurality of representative value obtainment modes that each represent a method for selecting or calculating, from the blood pressure data group, a representative value expressing the blood pressure data group measured during the identified instance, and setting one representative value obtainment mode from among the plurality of representative value obtainment modes; a representative value obtainment unit that selects or calculates the representative value for each day throughout the period from the blood pressure data group obtained by the blood pressure data obtainment unit, based on the representative value obtainment mode set by the representative value obtainment mode setting unit; and a display control unit that carries out control for displaying some or all of the representative values for each day throughout the period in the display screen.

In the present specification, “blood pressure data” refers generally to blood pressure values including a systolic blood pressure (SBP), which is a maximum blood pressure, and a diastolic blood pressure (DBP), which is a minimum blood pressure, as well as a pulse frequency, a number of measurement days, and so on.

The “instance” in the “first instance” and the “final instance” refers to a predetermined amount of time from when the blood pressure measurement has been started, such as ten minutes, for example. This is an amount of time assumed to be required for the measurement subject to accurately measure his/her blood pressure a plurality of times (two to three times, for example) using a blood pressure meter. Note that the blood pressure measurement in a given “instance” also includes automatically taking a plurality of blood pressure measurements within the stated predetermined amount of time. Specifically, this includes automatically measuring the blood pressure a plurality of times (three, for example) repeatedly in a period shorter than 10 minutes (a one-minute interval, for example) within the “predetermined amount of time” following the start of the blood pressure measurement.

With the blood pressure-related information display device according to one or more embodiments of the claimed invention, first, the blood pressure data obtainment unit obtains the blood pressure data from a measurement subject (typically a patient; the same applies hereinafter) measured each day throughout a given period. The representative value obtainment mode setting unit then identifies the first instance or the last instance, and presents, as a list in the display screen, a plurality of representative value obtainment modes expressing methods for selecting or calculating a representative value, representing a blood pressure data group measured during the identified instance, from the stated blood pressure data group. Then, one of the representative value obtainment modes is set by the representative value obtainment mode setting unit from among the plurality of representative value obtainment modes, in accordance with a selection made by a doctor, for example. The representative value obtainment unit selects or calculates the representative value for each day throughout the period from the blood pressure data group obtained by the blood pressure data obtainment unit, based on the representative value obtainment mode set by the representative value obtainment mode setting unit. The display control unit carries out control for displaying some or all of the representative values for each day throughout the period in the display screen.

When operating in this manner, in the blood pressure-related information display device according to one or more embodiments of the claimed invention, the doctor can set the representative value obtainment mode in accordance with his/her own preferences, from the plurality of representative value obtainment modes presented as a list in the display screen. As a result, the doctor can cause the representative values of blood pressure data selected in accordance with his/her own preferences to be displayed in the display screen.

In a blood pressure-related information display device according to one or more embodiments of the claimed invention, the plurality of representative value obtainment modes includes: a first representative value obtainment mode that selects, as the representative value, the blood pressure data measured first in the blood pressure data group measuring during the identified instance; a second representative value obtainment mode that selects, as the representative value, the blood pressure data measured second in the blood pressure data group measuring during the identified instance; a third representative value obtainment mode that selects, as the representative value, a minimum value in the blood pressure data group measuring during the identified instance; and a fourth representative value obtainment mode that calculates, as the representative value, an average value in which the blood pressure data group measuring during the identified instance has been averaged.

In the blood pressure-related information display device according to one or more embodiments of the claimed invention, the doctor can set a single representative value obtainment mode from among the stated first to fourth representative value obtainment modes. Accordingly, the doctor can more easily select the representative blood pressure value to serve as a basis for calculations from the plurality of measured blood pressure values, in accordance with his/her own preferences.

According to one or more embodiments of the claimed invention, the blood pressure-related information display device further includes a first average value calculation unit that calculates a first average value in which the representative values from each day in a first period of the stated period have been averaged, and the display control unit displays the first average value in the display screen.

In the blood pressure-related information display device according to one or more embodiments of the claimed invention, the display control unit displays the first average value in the display screen. Accordingly, the doctor can understand the state of the measurement subject's blood pressure, with ease and with certainty, by referring to the first average value in the display screen.

According to one or more embodiments of the claimed invention, the blood pressure-related information display device further includes a second average value calculation unit that calculates a second average value in which the representative values from each day in a second period of the stated period, different from the first period, have been averaged, and the display control unit displays the second average value alongside the first average value in the display screen.

In the blood pressure-related information display device according to one or more embodiments of the claimed invention, the display control unit displays the second average value alongside the first average value in the display screen. Accordingly, the doctor can easily compare the first average value and the second average value in the display screen, and therefore can understand the state of the measurement subject's blood pressure with further ease and certainty.

A non-transitory computer readable medium having a program stored thereon according to one or more embodiments of the claimed invention is a program that causes a computer to execute a method for displaying information related to a measurement subject's blood pressure in a display screen, the method including: a first step of obtaining blood pressure data of the measurement subject for each day in a given period; a second step of identifying a first instance from when the blood pressure data was measured first in a given day to after a predetermined amount of time has passed or a final instance from when the blood pressure data was measured last in a given day to a predetermined amount of time before that last measurement, presenting, as a list in the display screen, a plurality of representative value obtainment modes that each represent a method for selecting or calculating, from the blood pressure data group, a representative value expressing the blood pressure data group measured during the identified instance, and setting one representative value obtainment mode from among the plurality of representative value obtainment modes; a third step of selecting or calculating the representative value for each day throughout the period from the blood pressure data group obtained in the first step, based on the representative value obtainment mode set in the second step; and a fourth step of displaying some or all of the representative values selected or calculated in the third step in the display screen.

With the non-transitory computer-readable medium having the program stored thereon according to one or more embodiments of the claimed invention, a computer can be caused to execute a method for displaying information related to a measurement subject's blood pressure in a display screen in the following manner. That is, in the first step, blood pressure data of the measurement subject for each day in a given period is obtained. In the second step, the first instance or the last instance is identified, and a plurality of representative value obtainment modes expressing methods for selecting or calculating a representative value, representing a blood pressure data group measured during the identified instance, from the stated blood pressure data group are presented as a list in the display screen. Then, one representative value obtainment mode from among the plurality of representative value obtainment modes is set in accordance with an input from a doctor, for example. In the third step, the representative value for each day throughout the period is selected or calculated from the blood pressure data group obtained in the first step, based on the representative value obtainment mode set in the second step. In the fourth step, some or all of the representative values selected or calculated in the third step are displayed in the display screen.

In the case where a computer is caused to execute such a method, the doctor can set the representative value obtainment mode in accordance with his/her own preferences, from a plurality of representative value obtainment modes presented as a list in the display screen. As a result, the doctor can cause the representative values of blood pressure data selected in accordance with his/her own preferences to be displayed in the display screen.

### Advantageous Effects of Invention

As is clear from the foregoing descriptions, according to the blood pressure-related information display device and the non-transitory computer readable medium having the program stored thereon according to one or more embodiments of the claimed invention, a doctor can select a blood pressure value serving as a basis for calculations with ease, from a plurality of measured blood pressure values, in accordance with his/her own preferences.

### BRIEF DESCRIPTION OF DRAWINGS

FIG. 1 is a block diagram illustrating an embodiment in which a blood pressure-related information display device according to this invention is configured as a system on a network.

FIG. 2 is a block diagram illustrating the configuration of a hospital terminal that is a part of the aforementioned system.

FIG. 3 is a block diagram illustrating the configuration of a server that is a part of the aforementioned system.

FIG. 4 is a block diagram illustrating the configuration of a blood pressure meter that is a part of the aforementioned system.

FIG. 5 is a diagram illustrating an overview of a flow of operations performed by the server.

FIG. 6A is a diagram illustrating an array table for data of two morning instances, among blood pressure data stored in the server.

FIG. 6B is a diagram illustrating an array table for data of two evening instances, among blood pressure data stored in the server.

FIG. 7A is a diagram illustrating an array table for data of m morning instances, among blood pressure data stored in the server.

FIG. 7B is a diagram illustrating an array table for data of m evening instances, among blood pressure data stored in the server.

FIG. 8 is a diagram illustrating an example in which a representative value setting field, for setting a representative value obtainment mode, is displayed in a display screen of the hospital terminal.

FIG. 9 is a diagram illustrating an example in which fluctuations in a systolic blood pressure value and a diastolic blood pressure value, measured at mornings and evenings from approximately 60 days prior to a present examination day, are displayed as images in the display screen of the hospital terminal.

FIG. 10 is a diagram illustrating an example in which an average of blood pressure data in a previous examination period and an average of blood pressure data in a current examination period are displayed alongside each other as images in the display screen of the hospital terminal.

FIG. 11 is a diagram illustrating a flow of operations performed by the hospital terminal when functionality of the server is incorporated into the hospital terminal.

### DETAILED DESCRIPTION OF INVENTION

Hereinafter, embodiments of the invention will be described in detail with reference to the drawings.

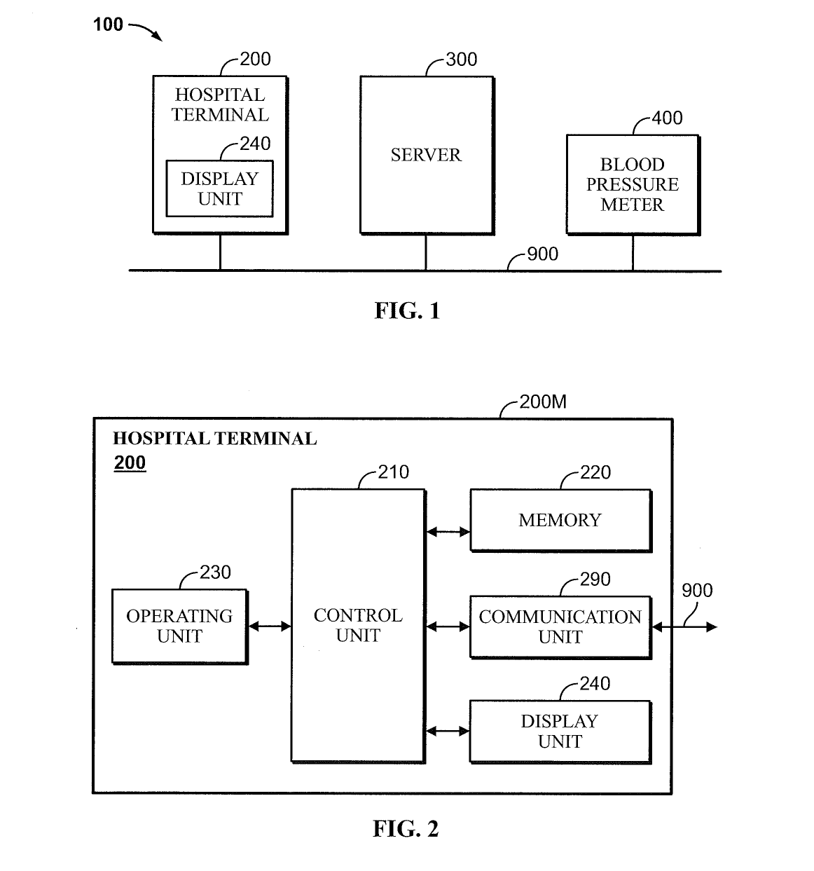

FIG. 1 illustrates an example in which a blood pressure-related information display device according to one or more embodiments of the claimed invention is embodied as a system on a network (indicated overall by reference numeral 100). The system 100 includes a hospital terminal 200 having a display unit 240 serving as a display screen, a server 300, and a blood pressure meter 400. The hospital terminal 200, the server 300, and the blood pressure meter 400 can communicate with each other over a network 900.

As shown in FIG. 2, the hospital terminal 200 includes a main body 200M, as well as a control unit 210, a memory 220, an operating unit 230, the display unit 240 serving as a display screen, and a communication unit 290, which are provided in the main body 200M. The hospital terminal 200 is configured of a commercially-available laptop computer in which application software (computer programs) for executing processes that will be mentioned later is installed, and is capable of accessing the server 300.

The control unit 210 includes a central processing unit (CPU) as well as auxiliary circuitry thereof, controls the various units of the hospital terminal 200, and executes processes that will be mentioned later in accordance with programs and data stored in the memory 220. In other words, the control unit 210 processes data inputted through the operating unit 230 and the communication unit 290, and stores the processed data in the memory 220, displays the processed data in the display unit 240, outputs the processed data from the communication unit 290, or the like.

The memory 220 includes a random access memory (RAM) used as a work region required by the control unit 210 to execute programs, and a read-only memory (ROM) for storing basic programs to be executed by the control unit 210. A semiconductor memory (a memory card, a solid-state drive (SSD)) or the like may be used as a storage medium in an auxiliary storage unit for complementing a storage region in the memory 220.

The operating unit 230 is in this example configured of a keyboard and a mouse, and inputs operation signals indicating operations performed by a user to a control unit 310. Instead of or in addition to the keyboard and the mouse, the operating unit 230 may be configured of another operating device, such as a touch panel.

The display unit 240 includes a display screen (for example, a liquid-crystal display (LCD) or an electroluminescence (EL) display). The display unit 240 displays a predetermined image in the display screen under the control of the control unit 210.

The communication unit 290 sends information from the control unit 210 to the server 300 via the network 900.

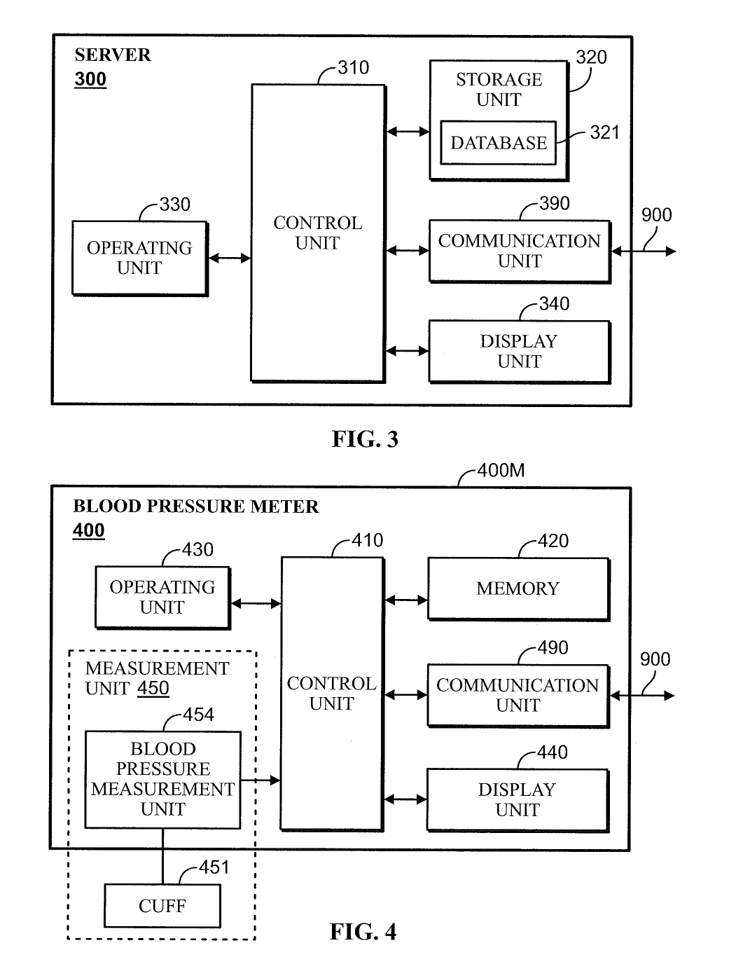

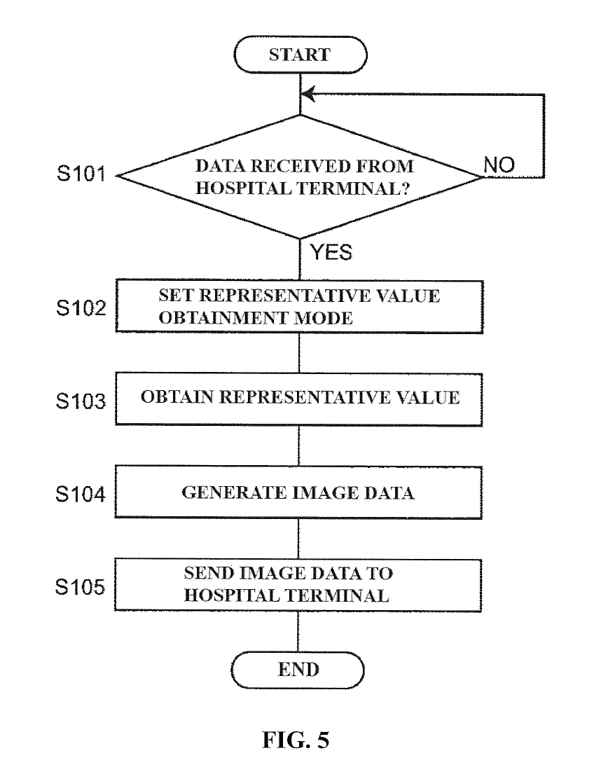

As shown in FIG. 3, the server 300 includes the control unit 310, a storage unit 320, an operating unit 330, a display unit 340, and a network communication unit 390. The server 300 is realized by installing programs (software) for executing processes that will be mentioned later in a generic computer device.

The control unit 310 includes a CPU and auxiliary circuitry thereof; the control unit 310 controls the various units of the server 300, executes predetermined processes in accordance with programs and data stored in the storage unit 320, processes data inputted from the operating unit 330 and the communication unit 390, stores processed data in the storage unit 320, displays processed data in the display unit 340, and outputs processed data from the communication unit 390.

The storage unit 320 includes a RAM used as a work region required by the control unit 310 to execute programs, and a ROM for storing basic programs to be executed by the control unit 310. A database 321 containing blood pressure measurement data sent from many users is provided in the storage unit 320. A magnetic disk (a hard disk (HD), a flexible disk (FD)), an optical disc (a compact disc (CD), a digital versatile disc (DVD), a Blu-ray disc (BD)), a magneto-optical disc (MO), or a semiconductor memory (a memory card, a solid state drive (SSD)), or the like may be used as a storage medium in an auxiliary storage unit for complementing the storage region of the storage unit 320.

The operating unit 330 is in this example configured of a keyboard and a mouse, and inputs operation signals indicating operations performed by a user to a control unit 310. Instead of or in addition to the keyboard and the mouse, the operating unit 330 may be configured of another operating device, such as a touch panel.

The display unit 340 includes a display screen (for example, an LCD or an EL display). The display unit 340 displays a predetermined image in the display panel under the control of the control unit 310.

The network communication unit 390 sends information from the control unit 310 to other apparatuses (in the present embodiment, the hospital terminal 200) via the network 900, and receives information sent via the network 900 from another apparatus and passes the information to the control unit 310.

As shown in FIG. 4, the blood pressure meter 400 includes a casing 400M, as well as a control unit 410, a memory 420, an operating unit 430, a display unit 440, a measurement unit 450 serving as a blood pressure data obtainment unit, and a communication unit 490, which are provided in the casing 400M.

In response to an operational device such as an operation button, an operation key, or the like (not shown) being manipulated by an operator, the operating unit 430 inputs operation signals based on the details of the operation to the control unit 410.

The measurement unit 450 includes a cuff 451 and a blood pressure measurement unit 454 for measuring a blood pressure, and obtains measured values of a systolic blood pressure (SBP), a diastolic blood pressure (DBP), and a pulse frequency from a patient.

The memory 420 stores data of programs for controlling the blood pressure meter 400, configuration data for configuring various types of functions of the blood pressure meter 400, measurement result data, and so on. The memory 420 is also used as a working memory when programs are executed.

The control unit 410 includes a CPU, and controls the memory 420, the display unit 440, and the communication unit 490 in accordance with programs stored in the memory 420 for controlling the blood pressure meter 400, in response to operation signals from the operating unit 430, and based on detection signals from the measurement unit 450.

The display unit 440 includes a display panel, indicators, and the like, and displays predetermined information in accordance with control signals from the control unit 410.

Under the control of the control unit 410, the communication unit 490 sends predetermined information to an external apparatus over the network 900, receives information from an external apparatus over the network 900 and passes the information to the control unit 410, and so on. The communication over the network 900 may be wireless or wired. Although the network 900 is the Internet in this embodiment, the network 900 is not limited thereto, and may be another type of network such as a local area network (LAN) installed in a hospital, one-to-one communication using a USB cable, or the like.

The system 100 is used as follows.

i) The measurement subject uses the blood pressure meter 400 to measure his/her own blood pressure on a daily basis over a given period, or in other words, over N days (where N is a natural number greater than or equal to 2). Specifically, it is assumed that the measurement subject measures his/her blood pressure three times in a predetermined period (ten minutes, in this example; called an “instance” hereinafter) from when the blood pressure measurement is started, and carries this out twice in the morning and the evening, respectively. In other words, the measurement subject measures two instances each day, in the morning and the evening, for a total of 12 blood pressure measurements.

The blood pressure meter 400 sends the measured blood pressure data to the server 300 via the network 900 along with a date/time of the measurement.

ii) Upon receiving the blood pressure data from the blood pressure meter 400 while in an operational state, the server 300 stores the received blood pressure data in the storage unit 320 along with the measurement date/time.

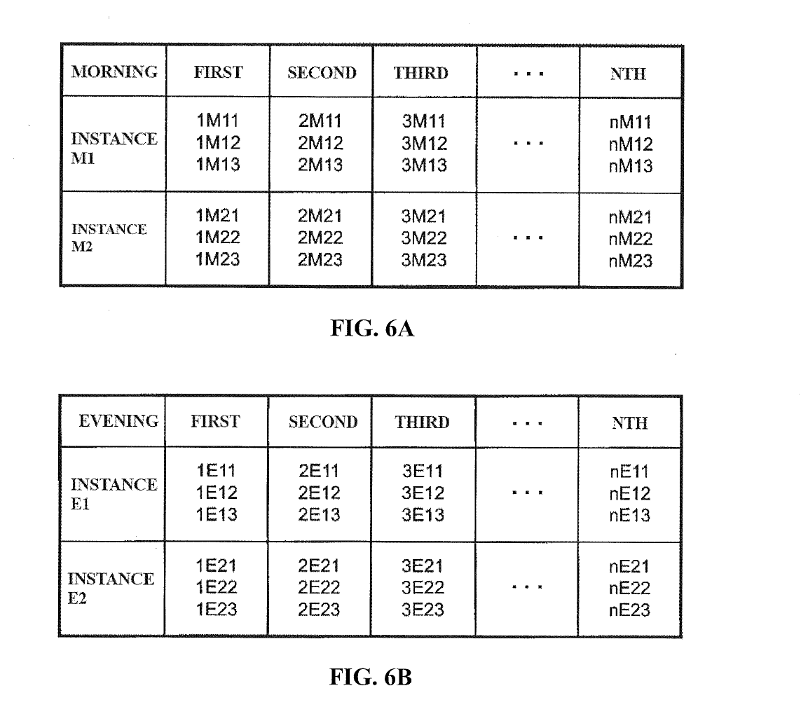

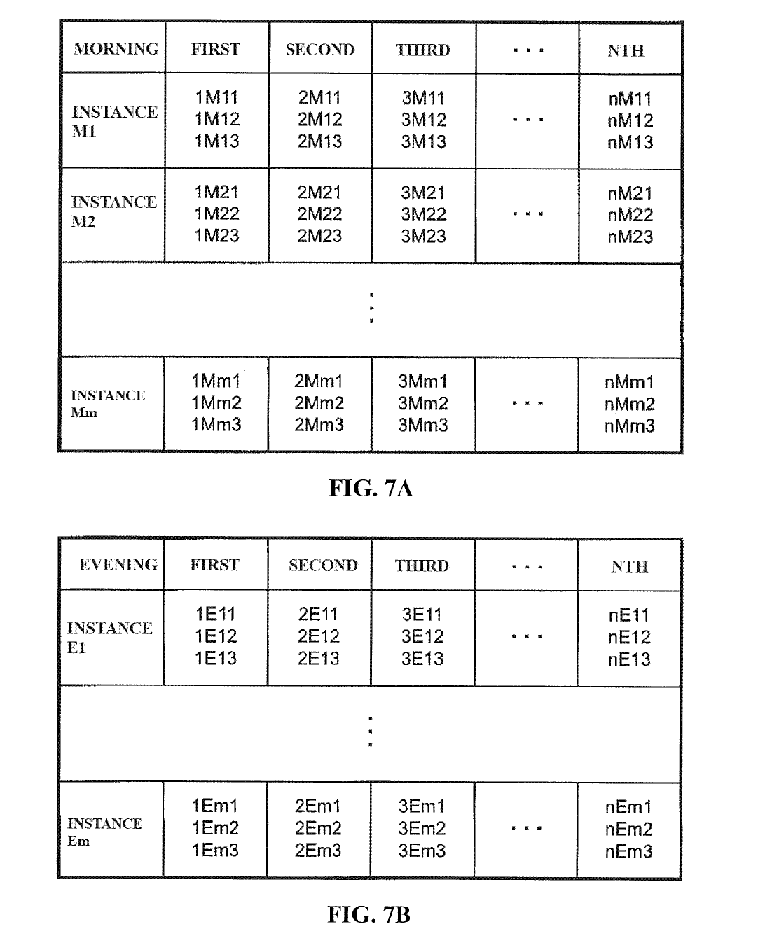

Here, the blood pressure data stored in the storage unit 320 of the server 300 is expressed as a data array table such as those shown in FIGS. 6A and 6B. In the data array tables shown in FIGS. 6A and 6B, an upper row identifies which day the blood pressure data belongs to, and a left-side column identifies which instance the blood pressure data belongs to (the same applies to FIGS. 7A and 7B).

FIG. 6A illustrates an array table of blood pressure data measured in the morning. For example, “1M23” in FIG. 6A expresses the third blood pressure data in a second instance M2 from the morning on the first day.

FIG. 6B illustrates an array table of blood pressure data measured in the evening. For example, “3E21” in FIG. 6B expresses the first blood pressure data in a second instance E2 from the evening on the third day.

Note that the measurement subject may measure the blood pressure in m instances (where m is a natural number of 2 or more) in both the morning and evening. In this case, the morning blood pressure data stored in the storage unit 320 of the server 300 is expressed as a data array table such as that shown in FIG. 7A. The evening blood pressure data is expressed as a data array table such as that shown in FIG. 7B.

iii) While the hospital terminal 200 is in an operational state, a doctor operates the operating unit 230 of the hospital terminal 200 and selects “blood pressure data display”, and the control unit 210 of the hospital terminal 200 requests a representative value obtainment mode to be set.

Here, the representative value obtainment mode refers to a method for identifying the first or last instance in a single day and selecting or calculating a representative value, representing a blood pressure data group measured during the identified instance, from that blood pressure data group. In this example, a first representative value obtainment mode that, for example, selects the first measured blood pressure data from the blood pressure data group (called a “first obtainment mode” hereinafter) serves as the representative value obtainment mode. There are also a second representative value obtainment mode that selects the second measured blood pressure data from the blood pressure data group (called a “second obtainment mode” hereinafter), a third representative value obtainment mode that selects a minimum value from the blood pressure data group (called a “minimum value obtainment mode” hereinafter), and a fourth representative value obtainment mode that calculates an average value for the blood pressure data group (called an “average value obtainment mode” hereinafter), serving as the representative value obtainment mode.

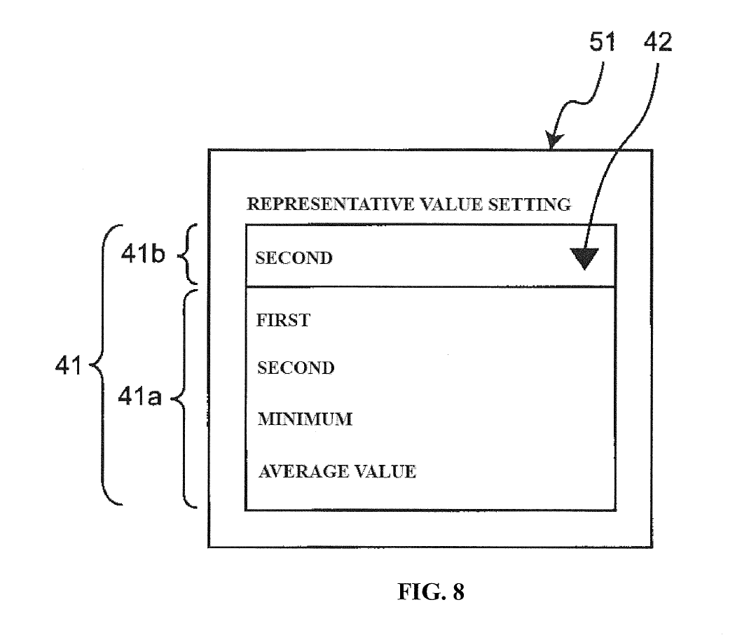

Specifically, as shown in FIG. 8, the control unit 210 of the hospital terminal 200 displays a representative value setting field 41, configured as a drop-down list, as an image in a part 51 of the display screen, for setting the representative value obtainment mode.

The representative value setting field 41 includes, in order from the top, a setting area 41b and a list area 41a that is displayed when a doctor clicks an inverted triangular mark 42 in the setting area 41b with the mouse.

“First”, representing the first obtainment mode, “second”, representing the second obtainment mode, “minimum value”, representing the minimum value obtainment mode, and “average value”, representing the average value obtainment mode, are displayed in the list area 41a in that order, from top to bottom, so that all of the representative value obtainment modes can be displayed as a list.

The list area 41a is configured so that any one of a plurality of types of representative value obtainment modes can be selected and set.

The representative value obtainment mode selected from the list area 41a is displayed in the setting area 41b (in this example, the “second” obtainment mode is selected in the list area 41a).

When the representative value obtainment mode is selected, the control unit 210 of the hospital terminal 200 sends, to the server 300, a request to obtain the blood pressure data, and sends the selected representative value obtainment mode along with date data indicating the current examination day to the server 300 as well.

iv) With the server 300 in an operational state, the control unit 310 of the server 300 stands by for the data including the representative value obtainment mode and the date data to be sent from the hospital terminal 200 via the network 900, as indicated by step S101 in FIG. 5. Upon receiving, via the communication unit 390, the data from the hospital terminal 200 over the network 900 (YES in step S101), the control unit 310 operates as a representative value obtainment mode setting unit, and sets the representative value obtainment mode based on the received data (step S102).

Next, the control unit 310 operates as a representative value obtainment unit, and selects or calculates, from the blood pressure data group stored in the storage unit 320 of the server 300, representative values for each day throughout the period, based on the representative value obtainment mode that has been set (step S103).

Specifically, in the aforementioned example, the control unit 310 selects, as a representative value, the second blood pressure data measured in the first instance in each day (indicated by 1M12, 2M12, and so on up to nM12 in FIG. 6A). Likewise, the control unit 310 selects, as a representative value, the second blood pressure data measured in the last instance in each day (indicated by 1E22, 2E22, and so on up to nE22 in FIG. 6B). Here, the representative values include a representative blood pressure value of the blood pressure values and a representative pulse frequency of the pulse frequencies.

Furthermore, the control unit 310 obtains, for both the morning and the evening, each day's representative value as well as a number of measurement days in a period from the last examination to the current examination (a current examination period serving as a first period). Then, operating as a first average value calculation unit, the control unit 310 calculates a current examination average value, serving as a first average value in which the representative values in the current examination period have been averaged, based on the obtained representative values and the number of measurement days. Here, the current examination average value includes an average blood pressure value of the blood pressure values and an average pulse frequency of the pulse frequencies.

Likewise, the control unit 310 obtains, for both the morning and the evening, each day's representative value as well as a number of measurement days in a period from the second-to-last examination to the last examination (a previous examination period serving as a second period). Then, operating as a second average value calculation unit, the control unit 310 calculates a previous examination average value, serving as a second average value in which the representative values in the previous examination period have been averaged, based on the obtained representative values and the number of measurement days. Here, the previous examination average value includes an average blood pressure value of the blood pressure values and an average pulse frequency of the pulse frequencies.

Specifically, in the above example, a current examination average blood pressure value (a set of the SBD and the DBP, indicated as SBP/DBP; the same applies hereinafter) is 138/89 mmHg for both morning and evening. On the other hand, a previous examination average blood pressure value is 140/90 mmHg for both morning and evening.

Furthermore, in the above example, a current examination average pulse frequency is 64 bpm for both morning and evening. On the other hand, a previous examination average pulse frequency is 65 bpm for both morning and evening.

Furthermore, in this example, a number of measurement days in the current examination period is 14 days for both morning and evening. On the other hand, a number of measurement days in the previous examination period is 12 days for both morning and evening.

Next, the control unit 310c generates image data (described in more detail later) to be displayed in the display unit 240 of the hospital terminal 200 based on the representative values, the average values of the blood pressure data from the current examination and the last examination, and so on (step S104 in FIG. 5).

Next, the control unit 310 stores the generated image data in the database 321. The image data is also sent from the communication unit 390 to the hospital terminal 200 over the network 900 (step S105).

v) The hospital terminal 200 receives the image data from the communication unit 290 over the network 900 and stores the image data in the memory 220. Then, operating as a display control unit, the control unit 210 of the hospital terminal 200 displays the image data stored in the memory 220 in the display screen of the display unit 240.

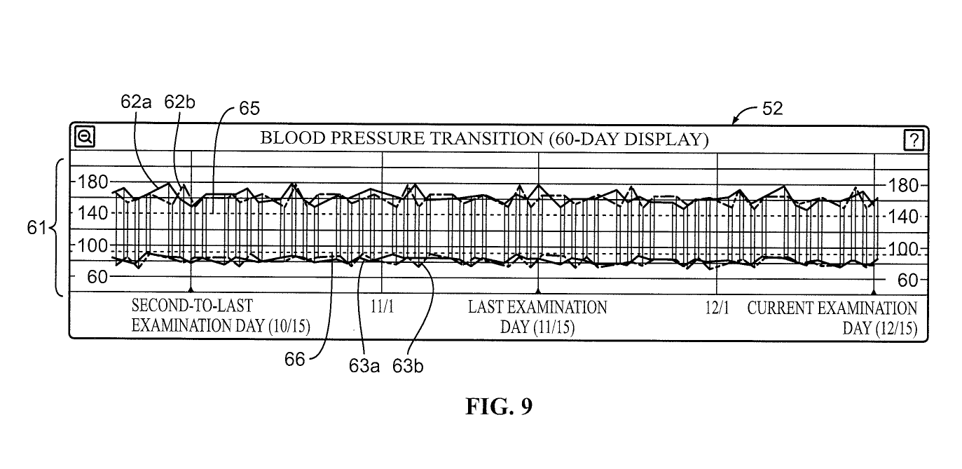

An image displayed in a part 52 of the display screen in the display unit 240 is as indicated in FIG. 9, for example. As shown in FIG. 9, a blood pressure transitive graph display field 61, indicating a transition of the measurement subject's representative blood pressure value over time, is displayed as an image.

In the blood pressure transitive graph display field 61, the measurement subject's representative blood pressure values for each day in an approximately 60-day period prior to the current examination day (12/15 (December 15) in this example) are displayed as polygonal line graphs 62a, 62b, 63a, and 63b, where the horizontal axis represents time (unit: days) and the vertical axis represents the blood pressure value (unit: mmHg).

Here, the polygonal line 62a indicates the SBP for the morning representative blood pressure values, whereas the polygonal line 62b indicates the SBP for the evening representative blood pressure values. Likewise, the polygonal line 63a indicates the DBP for the morning representative blood pressure values, whereas the polygonal line 63b indicates the DBP for the evening representative blood pressure values. Accordingly, using the display unit 240, the doctor can instinctively understand the transition of the measurement subject's representative blood pressure values over time in the approximately 60 days prior to the current examination day (12/15).

The blood pressure transitive graph display field 61 also includes broken lines 65 and 66 that extend straight in the horizontal direction. The broken line 65 indicates 140 mmHg as a target value for the SBP set by the measurement subject or a doctor in charge, whereas the broken line 66 indicates 90 mmHg as a target value for the DBP set by the measurement subject or the doctor in charge. Accordingly, by comparing the polygonal line graphs 62a, 62b, 63a, and 63b to the broken lines 65 and 66, the doctor can determine at a glance whether or not the measurement subject's blood pressure values have met the target values set as described above.

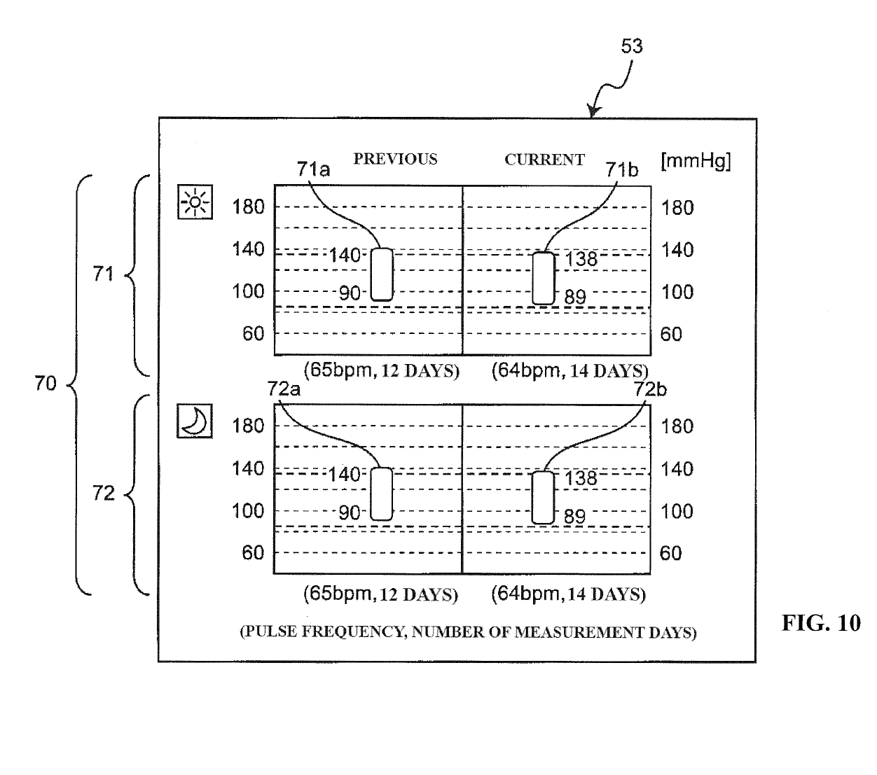

An image displayed in a part 53 of the display screen in the display unit 240 is as indicated in FIG. 10, for example. Specifically, as shown in FIG. 10, an average blood pressure graph display field 70 is displayed as an image.

The average blood pressure graph display field 70 includes a morning average blood pressure graph display field 71 and an evening average blood pressure graph display field 72, in that order from the top.

In the morning average blood pressure graph display field 71, a floating column chart 71b indicating the morning average blood pressure value in the current examination, with the SBP on top and the DBP on the bottom, is displayed with the vertical axis representing a blood pressure value (unit: mmHg), and is displayed along with the SBP and DBP values. Likewise, in the evening average blood pressure graph display field 72, a floating column chart 72b indicating the evening average blood pressure value in the current examination, with the SBP on top and the DBP on the bottom, is displayed with the vertical axis representing a blood pressure value (unit: mmHg), and is displayed along with the SBP and DBP values. Through this, the doctor can understand the state of the measurement subject's blood pressure, with ease and with certainty, by referring to the average blood pressure values in the part 53 of the display screen.

Meanwhile, in the morning average blood pressure graph display field 71, a floating column chart 71a indicating the morning average blood pressure value in the last examination, with the SBP on top and the DBP on the bottom, is displayed along with the SBP and DBP values. Likewise, a floating column chart 72a indicating the evening average blood pressure value in the last examination, with the SBP on top and the DBP on the bottom, is displayed along with the SBP and DBP values. These floating column charts 71a and 72a are displayed alongside the floating column charts 71b and 72b, to the left thereof. Through this, the doctor can easily compare the average blood pressure values from the current examination with the average blood pressure values from the last examination in the display screen, and can thus understand the state of the measurement subject's blood pressure with greater ease and certainty.

Furthermore, corresponding average pulse frequencies and numbers of measurement days are displayed below the floating column charts 71a, 71b, 72a, and 72b, respectively.

When operating in this manner, according to the system 100, the doctor can set the representative value obtainment mode in accordance with his/her own preferences, from a plurality of representative value obtainment modes. As a result, the doctor can cause the representative values of blood pressure data selected in accordance with his/her own preferences to be displayed in the display screen.

Note that the control unit 310 of the server 300 may send only data for generating an image to the hospital terminal 200, rather than sending the image data, and the control unit 210 of the hospital terminal 200 may then generate the image itself.

Furthermore, although this embodiment describes the “first obtainment mode”, the “second obtainment mode”, the “minimum value obtainment mode”, and the “average value obtainment mode” as the representative value obtainment modes, the representative value obtainment modes are not limited thereto. For example, the configuration may be such that the details of the representative value obtainment mode can be set in accordance with the doctor's preferences, such as by calculating an average value in which the blood pressure data measured first in the first and second instances in the morning are averaged.

Furthermore, although the control unit 310 calculates the average value of the representative values in the current examination period, the last examination period, and so on for both morning and evening, embodiments of the invention are not limited thereto. For example, an average value of the representative values in another period may be calculated, such as in a period from an examination carried out four times ago to an examination carried out three times ago (a thrice-previous examination period), a period from prior to an initial examination to an initial examination (an initial examination period), or the like. An average value of the representative values over a five-day period from the current examination may be calculated as well.

Furthermore, although the measurement subject measures his/her own blood pressure on a daily basis in this embodiment, embodiments of the invention are not limited thereto. For example, the measurement frequency may be gradually increase, by measuring the blood pressure once every two days at the beginning of the stated period, then measuring the blood pressure every day on weekdays, and so on.

Furthermore, although the measurement subject measures his/her own blood pressure each day at two instances in the morning and evening, respectively, in this embodiment, embodiments of the invention are not limited thereto. For example, s/he may measure three instances of the blood pressure in the morning and one instance of the blood pressure in the evening.

Furthermore, although the measurement subject measures his/her own blood pressure three times in a single instance in this embodiment, embodiments of the invention are not limited thereto. For example, s/he may measure the blood pressure once or twice in a single instance, or may measure the blood pressure four or more times in a single instance. Likewise, for example, s/he may measure the blood pressure twice in a single instance in the morning, and may measure the blood pressure four times in a single instance in the evening.

Furthermore, although the blood pressure-related information display device according to one or more embodiments of the present invention is configured of a system 100 including a hospital terminal 200, a server 300, and a blood pressure meter 400 connected over a network in this embodiment, embodiments of the invention are not limited thereto.

For example, the blood pressure-related information display device according to one or more embodiments of the present invention may be configured of only the blood pressure meter 400 and the hospital terminal 200. That is, in addition to the functions originally performed by the control unit 210, memory 220, the operating unit 230, the display unit 240, and the communication unit 290 in the hospital terminal 200, these units may also carry out the functions of the control unit 310, the storage unit 320, the operating unit 330, the display unit 340, and the network communication unit 390 in the server 300, respectively. In this case, a program for causing the control unit 210 to execute a method that displays information related to the measurement subject's blood pressure in the display screen, as will be described below, is installed in the memory 220 of the hospital terminal 200. Through this, the blood pressure-related information display device according to one or more embodiments of the present invention can be configured as a small-sized, compact unit.

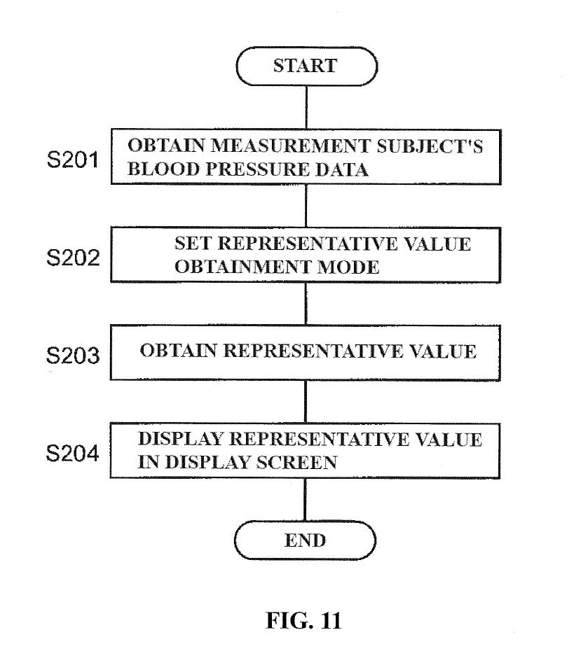

The stated method can be broadly divided in four steps, as indicated in FIG. 11.

In a first step S201 shown in FIG. 11, the measurement subject's blood pressure data, measured by the blood pressure meter 400, is obtained.

In a second step S202, the first instance or the last instance is identified; a plurality of representative value obtainment modes expressing methods for selecting or calculating a representative value, representing the blood pressure data group measured during the identified instance, from the stated blood pressure data group are presented; and one of the representative value obtainment modes is then set. This second step S202 corresponds to step S102 in FIG. 5.

In a third step S203, the representative values are selected or calculated for each day throughout the period from the blood pressure data group obtained in the first step S201, based on the representative value obtainment mode set in the second step S202. This third step S203 corresponds to step S103 in FIG. 5.

In a fourth step S204, the blood pressure transitive graph display field 61, indicating the transition over time of the representative values for the measurement subject's blood pressure as selected in the third step, is displayed in the part 51 of the display screen.

In the case where a computer is caused to execute such a method, the doctor can set the representative value obtainment mode in accordance with his/her own preferences, from a plurality of representative value obtainment modes. As a result, the doctor can cause the representative values of blood pressure data selected in accordance with his/her own preferences to be displayed in the display screen.

This program can be recorded onto a recording medium such as a CD, a DVD, a flash memory, or the like as application software. By being installed in what is substantially a computer device, such as a hospital terminal, a personal computer, a personal digital assistant (PDA), a smartphone, or the like, the application software recorded onto the recording medium can cause the computer device to execute the method for displaying the information related to the measurement subject's blood pressure in the display screen.

Note that the blood pressure-related information display device according to one or more embodiments of the present invention may substantially be configured only of the hospital terminal by further incorporating the functionality of the measurement unit 450 of the blood pressure meter 400 into the hospital terminal (into which the functionality of the server 300 has already been incorporated).

Meanwhile, instead of using these types of hospital terminals (into which the functionality of the server 300 has been incorporated, and into which the measurement unit 450 of the blood pressure meter 400 has further been incorporated), the blood pressure-related information display device according to one or more embodiments of the present invention may be configured using a smartphone or mobile telephone having the same functions as the constituent elements of the respective types of hospital terminals.

### REFERENCE NUMERALS LIST

100 system

200 hospital terminal

240 display unit

300 server

400 blood pressure meter

1. A blood pressure-related information display device that displays information related to a blood pressure of a measurement subject in a display screen, the device comprising:
a blood pressure data obtainment unit that obtains blood pressure data of the measurement subject for each day in a given period;
a representative value obtainment mode setting unit that identifies a first instance from when the blood pressure data was measured first in a given day to after a predetermined amount of time has passed or a final instance from when the blood pressure data was measured last in a given day to a predetermined amount of time before that last measurement, presenting, as a list in the display screen, a plurality of representative value obtainment modes that each represent a method for selecting or calculating, from the blood pressure data group, a representative value expressing the blood pressure data group measured during the identified instance, and setting one representative value obtainment mode from among the plurality of representative value obtainment modes;
a representative, value obtainment unit that selects or calculates the representative value for each day throughout the period from the blood pressure data group obtained by the blood pressure data obtainment unit, based on the representative value obtainment mode set by the representative value obtainment mode setting unit; and
a display control unit that carries out control for displaying some or all of the representative values for each day throughout the period in the display screen.

2. The blood pressure-related information display device according to claim 1,
wherein the plurality of representative value obtainment modes comprises:
a first representative value obtainment mode that selects, as the representative value, the blood pressure data measured first in the blood pressure data group measuring during the identified instance;
a second representative value obtainment mode that selects, as the representative value, the blood pressure data measured second in the blood pressure data group measuring during the identified instance;
a third representative value obtainment mode that selects, as the representative value, a minimum value in the blood pressure data group measuring during the identified instance; and
a fourth representative value obtainment mode that calculates, as the representative value, an average value in which the blood pressure data group measuring during the identified instance has been averaged.

3. The blood pressure-related information display device according to claim 1, further comprising:
a first average value calculation unit that calculates a first average value in which the representative values from each day in a first period of the stated period have been averaged,
wherein the display control unit displays the first average value in the display screen.

4. The blood pressure-related information display device according to claim 3, further comprising:
a second average value calculation unit that calculates a second average value in which the representative values from each day in a second period of the stated period, different from the first period, have been averaged,
wherein the display control unit displays the second average value alongside the first average value in the display screen.

5. A non-transitory computer readable medium having a program stored thereon that causes a computer to execute a method for displaying information related to a measurement subject's blood pressure in a display screen, the method comprising:
a second step of identifying a first instance from when the blood pressure data was measured first in a given day to after a predetermined amount of time has passed or a final instance from when the blood pressure data was measured last in a given day to a predetermined amount of time before that last measurement, presenting, as a list in the display screen, a plurality of representative value obtainment modes that each represent a method for selecting or calculating, from the blood pressure data group, a representative value expressing the blood pressure data group measured during the identified instance, and setting one representative value obtainment mode from among the plurality of representative value obtainment modes;
a third step of selecting or calculating the representative value for each day throughout the period from the blood pressure data group obtained in the first step, based on the representative value obtainment mode set in the second step; and
a fourth step of displaying some or all of the representative values selected or calculated in the third step in the display screen.

6. The blood pressure-related information display device according to claim 2, further comprising:
a first average value calculation unit that calculates a first average value in which the representative values from each day in a first period of the stated period have been averaged,
wherein the display control unit displays the first average value in the display screen.

7. The blood pressure-related information display device according to claim 6, further comprising:
a second average value calculation unit that calculates a second average value in which the representative values from each day in a second period of the stated period, different from the first period, have been averaged,
wherein the display control unit displays the second average value alongside the first average value in the display screen.
